(19) United States
(12) Patent Application Publication
(10) Pub. No.: US 2021/0177745 A1
(43) Pub. Date: Jun. 17, 2021

(54) DISINFECTING COMPOSITION, APPLICATOR, AND METHOD OF DISINFECTING

(71) Applicant: Medline Industries, Inc., Northfield, IL (US)

(72) Inventors: Joseph Bryzek, JR., Kenosha, WI (US); Brett Fortin, Bloomfield Hills, MI (US); Martin Coffey, Buffalo Grove, IL (US); Sharbel Maalouf, Pleasant Prairie, WI (US); Yevgeniya Bala, Gurnee, IL (US)

(21) Appl. No.: 16/717,610

(22) Filed: Dec. 17, 2019

(51) Int. Cl. A61K 9/00; A61K 31/045; A61K 47/44; A61K 31/23; A61K 47/18; A61K 47/10; A61K 47/22; A61M 31/00

(52) U.S. Cl. CPC A61K 9/0043; A61K 31/045; A61K 47/44; A61K 31/23; A61M 2209/06; A61K 47/10; A61K 47/22; A61M 31/00; A61M 2210/0618; A61K 47/18

(57) ABSTRACT

A disinfecting composition includes at least one alcohol and at least one emollient in amount effective to reduce a stinging sensation produced by contacting a wall of a nasal passage with the alcohol. An applicator generally includes an implement to apply a disinfecting composition to a nasal passage of a patient. A method of disinfecting a nasal passage of a patient includes applying a disinfecting composition to the nasal passage of the patient.

### TECHNICAL FIELD

The disclosure is in the field of patient care products and in various non-exclusive embodiments is directed to a disinfecting composition, applicator, and method for disinfecting a nasal passage of a patient. In some embodiments the disclosure provides a packaged applicator system.

### BACKGROUND

In patient care, cleaning and disinfecting agents are frequently used in pre- and post-surgical treatments. For example, swabsticks (or “swabs”) containing 10% povidone-iodine disinfecting solution are available commercially. These swabs can be used in first aid applications to help prevent infection in minor cuts, scrapes and burns, and in other medical applications. An exemplary usage of povidone-iodine swabs is to clean the nasal passages prior to surgery. Disinfecting of the nasal passages is believed to reduce the incidence of post-surgical infections such as Methicillin Resistant Staphylococcus aureus (MRSA).

It would be beneficial to provide nasal swabs containing a disinfectant composition including alcohol. Use of alcohol to disinfect the nasal passages is known, but the alcohol in the disinfectant composition can create an unpleasant stinging sensation when applied to sensitive nasal tissues. One known alcohol-based nasal disinfectant system includes an emollient which is added with the goal of reducing the stinging sensation. The emollient is a mixture of oils and is provided in an ampule. In use, the ampule is shaken to yield a suspension of the oil in the alcohol, and the tip of the ampule is crushed. The swab tip is wetted with the solution and used to clean the nasal passages.

It has now been found that a swab that includes a lower alcohol and an emollient that is miscible with alcohol can be provided. The swab is useful in disinfecting the nasal passages prior to surgery. The alcohol and emollient together form a solution that is stable and does not require pre-mixing, such that the swab can be provided pre-soaked with the disinfecting solution in a sealed foil or polymeric package without the need for an ampule or any pre-mixing prior to administration. The emollient may be castor oil, and the disinfectant composition may include other ingredients such as cooling agents, additional emollients, fragrances, and preservatives. Methods of administration also are provided. Also provided are an applicator for disinfecting a nasal passageway of a patient with a disinfecting composition, and a packaged applicator system.

### BRIEF DESCRIPTION OF THE DRAWINGS

FIG. 1 is a perspective view of an embodiment of a packaged applicator system as supplied to a clinician and prior to opening;

FIG. 2 is a perspective view of the packaged applicator system of FIG. 1 in an opened state; and

FIG. 3 is a perspective view of one of the swabs of the packaged applicator system.

### DETAILED DESCRIPTION

It has now been found that patient comfort can be increased by providing a nasal swab including a disinfecting composition, the disinfecting composition comprising at least one C₁-C₃ alcohol and at least one emollient. The emollient should be present in amount effective to reduce a stinging sensation produced when the alcohol contacts a wall of a nasal passage with the alcohol. The emollient is miscible with the alcohol such that at least one emollient and the alcohol form a solution, and the entire disinfecting composition may be a single-phase composition. The disinfecting composition is applied by a caregiver or by the patient himself or herself via a swab.

The disinfecting composition generally includes any one or more suitable C₁-C₃ alcohols in any amount suitable to reduce the number of active pathogens in a nasal passage that is contacted with the disinfecting composition. The disinfecting composition in exemplary embodiments comprises alcohol in an amount of at least 40, 45, 50, 55, 60, 65 or 70%, and optionally in an amount ranging from 40 to 80, 45 to 75, 50 to 70, or 55 to 65%, all percentages by weight of the disinfecting composition. In some embodiments a disinfecting composition includes at least one C₁-C₃ alcohol. Examples of C₁-C₃ alcohols include any one or more of methyl alcohol, ethyl alcohol, n-propyl alcohol, and isopropyl alcohol. In some embodiments, the disinfecting composition comprises ethyl alcohol.

The disinfecting composition can optionally include a denaturant to inhibit improper ingestion of the composition. Any suitable denaturant can be included in a disinfecting composition and when used the denaturant may be present in any suitable amount. An exemplary denaturant is isopropyl alcohol. In some exemplary embodiments, the disinfecting composition comprises a denaturant in an amount ranging from 1 to 10, 2 to 7, 2.5 to 5% by weight of the disinfecting composition. In some embodiments, a disinfecting composition comprises ethyl alcohol and a lesser amount of isopropyl alcohol as a denaturant. Other denaturants may be present in amounts suitable to render the composition unpalatable. Exemplary denaturants include denatonium benzoate and t-butyl alcohol.

The composition also generally includes one or more emollients in amount effective to reduce a stinging sensation produced by contacting a wall of a nasal passage with the alcohol included the disinfecting composition. The emollient can have a viscosity that is sufficient to retain a disinfecting composition within a nasal passage for a period of time sufficient to increase inactivation of pathogens. In some aspects, useful emollients have viscosities at 10° C. ranging from 1 to 5000 cP, such as 500 to 3500 cP, 1000 to 3000 cP, or 1500 to 2500 cP. In any case, the emollient may be present in any amount suitable to reduce the stinging sensation of the alcohol in the nasal passages. While this amount may vary from individual to individual, the emollient can be present in an amount of at least 10%, at least 15%, at least 20%, at least 25%, at least 30%, at least 35%, at least 40%, at least 45%, and at least 50%, and optionally amounts ranging from 10 to 50%, 15 to 45%, 20 to 40%, and 25 to 35%, all percentages by weight of the disinfecting composition.

Castor oil is deemed particularly suitable as an emollient that is miscible with alcohol. Other examples of useful emollients include any one or more of fatty acids such as behenic acid, caprylic acid, lauric acid, myristic acid, oleic acid, palmitic acid, ricinoleic acid, and stearic acid; fatty esters such as glyceryl laurate, glyceryl stearate, isopropyl myristate, and isopropyl palmitate; fatty alcohols such as cetearyl alcohol, cetyl alcohol, lauryl alcohol, oleyl alcohol, stearyl alcohol, and derivatives thereof. It is generally thought that miscibility of the emollient in the one or more alcohols can aid with miscibility of other components in the disinfecting composition.

The disinfecting composition can optionally include an additional emollient, such as one or more of the emollients listed above, in amounts that are miscible with the alcohol and the first emollient. In some embodiments, the disinfecting composition includes an additional emollient in an amount ranging from 0.1 to 2.0, 0.1 to 1.5, 0.5 to 1.5, or 0.7 to 0.9% by weight of the disinfecting composition.

The disinfecting composition can also optionally include one or more diluents. In some embodiments, the one or more diluents can be included in an amount ranging from 1 to 30, 2 to 20, 3 to 15, 4 to 13, 7 to 10% by weight of the disinfecting composition. Examples of useful diluents include water (e.g. purified USP), glycerin, and propylene glycol.

The disinfecting composition can also optionally include a cooling agent to provide a cooling sensation in a nasal passage. It is generally thought that a cooling sensation imparted by cooling agent can detract from stinging sensations caused by contacting nasal tissues with alcohol. The cooling agents can be present in any suitable total amount; exemplary amounts of cooling agents can from 0.01 to 0.15%, such as 0.02 to 0.10%, 0.03 to 0.08%, or 0.04 to 0.06%, by weight of the disinfecting composition. Useful cooling agents include any one or more of menthol, peppermint oil, icilin, sugar alcohols such as xylitol, and mixtures and derivatives thereof.

The disinfecting composition may optionally further include any one or more of fragrances, colors, preservatives, antioxidants, humectants, and other additives. These ingredients may be added in any amounts suitable for their intended purposes.

For example, any suitable fragrance can be included in a disinfecting composition. The fragrance may be an oil or contain an oil that is immiscible or not fully miscible with the alcohol/emollient solution. This is because the fragrance is typically used in a small amount (less than 5% and sometimes less than 1% by weight of the composition) and the fragrance will be perceived by the clinician and the patient even if the fragrance has separated from the other components of the disinfecting composition. In other embodiments, the fragrance is miscible and the disinfecting composition is a single-phase composition in its entirety. Examples of useful fragrances include oils such as aloe oil, bergamot oil, cinnamon oil, clary sage oil, citrus oil, clove oil, eucalyptus oil, ginger oil, grapefruit oil, lavender oil, lemon oil, lime oil, mandarin oil, orange oil, oregano oil, peppermint oil, petitgrain oil, rosemary oil, sandalwood oil, spearmint oil, tangerine oil, tea tree oil, thyme oil, ylang oil, and other fragrant oils. Additionally, the disinfecting composition can comprise an any suitable antioxidant. Examples of useful antioxidants include butylated hydroxytoluene (BHT), vitamin A, vitamin B3, and vitamin E, and their derivatives. Examples of other suitable additives include vitamin C and ascorbyl palmitate. When present, the antioxidant may be present in any amount suitable to provide an antioxidant effect, typically less than 0.5% by weight of the cleansing composition. Similarly, the disinfecting composition can generally include any suitable preservative. Examples of suitable preservatives include benzyl alcohol and polyaminopropyl biguanide. The disinfecting composition can include one or more preservatives in a total amount ranging from 0.05 to 1.5, 0.1 to 1, and 0.2 to 0.8% weight of the disinfecting composition.

Examples of useful humectants include any one or more of collagen, a polyhydric alcohol, urea, etc. Useful polyhydric alcohols include butylene glycol, glycerin, hexylene glycol, polyalkylene glycols such as polyethylene glycol and polypropylene glycol, propylene glycol, dipropylene glycol, sorbitol, triethylene glycol, arylpropyne glycol, alkaline polyols 1,3-dibutylene glycol, allantoin, derivatives and mixtures thereof, etc.

The disinfecting composition may be entirely a single-phase composition, i.e., a solution in which none of the components separate during transport and storage. It is also contemplated that some portion of the composition, up to 5% but preferably less than 4, less than 3%, less than 2%, or less than 1%, may comprise a second phase, this second phase being principally fragrance components. As noted above, it is contemplated that the function of the composition will not be impaired if the fragrant component separate somewhat during shipping and storage.

The composition is intended to have a disinfecting property. One test for whether the composition has a disinfecting property is to measure its efficacy against MRSA in vitro. An exemplary testing protocol is ASTM E2315-16 “Standard Guide for Assessment of Antimicrobial Activity Using a Time Kill Procedure” in accordance with 21 CFR Part 58 “Good Laboratory Practices for Nonclinical Laboratory Studies.” Under this protocol, the composition should exhibit a log₁₀ reduction of MRSA of at least 1.0, preferably at least 1.25, more preferably at least 1.5, more preferably at least 1.75, and more preferably at least 2.0, after 15 seconds. Also under this protocol, the composition should exhibit a log₁₀ reduction of MRSA of at least 3.5, preferably at least 4.0, more preferably at least 4.5, more preferably at least 5.0, and more preferably at least 5.5, after eight hours, and a log₁₀ reduction of MRSA of at least 4.5, preferably at least 5.0, more preferably at least 5.5, more preferably at least 6.0, and more preferably at least 6.5, after twelve hours.

While the composition may be supplied as a liquid for direct application to the nasal passages, more typically it is contemplated that the composition will be supplied with an applicator. The applicator can generally include an implement capable of applying a disinfecting composition to a nasal passage of a patient. The applicator may comprise an absorbent material and a disinfecting composition disposed on the absorbent material. The absorbent material can generally include any one or more of natural and synthetic materials capable of applying a disinfecting composition to a nasal passage of a patient, and may include fibers or foams that can be used individually or in combination. Fibers can generally be used in any configuration such as any one or more of wound, woven, non-woven, layered, carded, blended, etc. Useful examples of absorbent materials include any one or more of cotton, rayon, polyester, polyethylene, polyurethane, etc.

The absorbent material is sized to fit within a nasal passage of a patient. In some embodiments, an applicator comprises a swab including an absorbent material provided on an end of an applicator stick and a disinfecting composition disposed on the absorbent material. The applicator stick can be generally formed of any material such as one or more of wood, wound paper, wire, polymers such as polypropylene, polyethylene, etc. In other embodiments, an applicator can comprise a reservoir for retaining a disinfecting composition. Useful applicators comprising a reservoir include a spray bottle, an atomizer, an irrigation syringe, a vial, ampoule, etc. In yet other aspects, an applicator can comprise materials such as cloth, gauze, a cotton ball, etc.

Generally, the applicator can be packaged in any useful manner. In some aspects, an applicator can be packaged in a sealed container as part of a packaged applicator system. The sealed container is provided to inhibit evaporation of a disinfecting composition and contamination of the applicator. Any suitable container is contemplated, and exemplary containers can comprise one or more of a polymer film, foil, paper, paperboard, and cardboard.

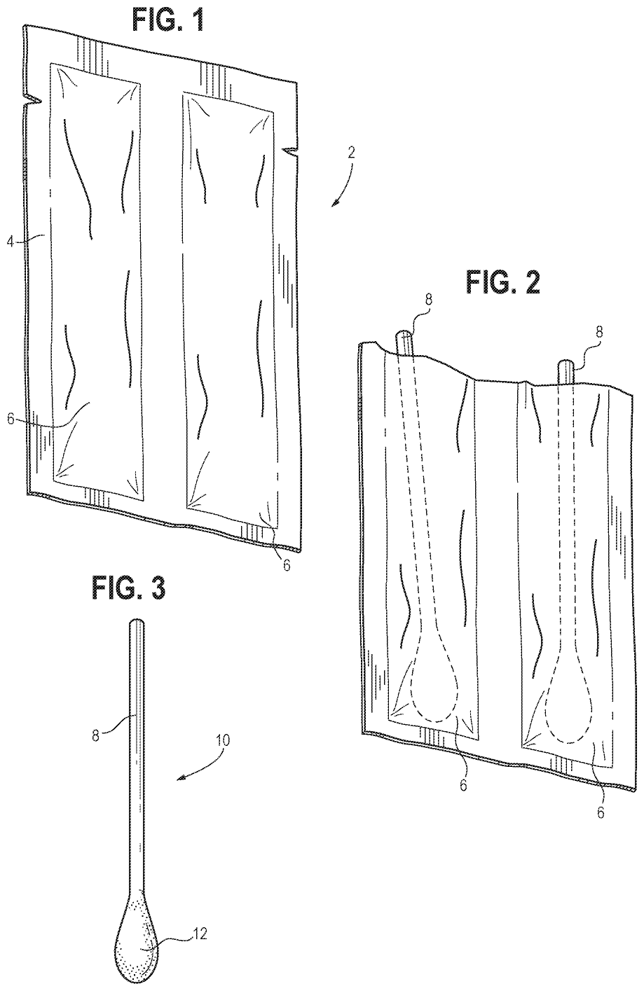

The container can generally include any number of applicators, but it is deemed particularly useful to package the applicator in pairs, one for each nostril of a patient. For example, two swabs can be packaged together within a sealed container or separately within individually sealed compartments of a sealed container. For example, FIG. 1 depicts an embodiment of a packaged applicator system 2 including a sealed film container 4 including two separate compartments 6. The compartments 6 each enclose a single swab. The package is designed to be torn open by a clinician or patient to access the swabs therewithin. FIG. 2 shows the container of FIG. 1 in an opened state with a portion of the container removed to expose ends of swab sticks 8 of the swabs. FIG. 3 depicts a swab 10 including a swab stick 8 and a disinfecting composition as described herein soaked within the absorbent material 12.

Also provided herein are method of making an applicator, a method of packaging an applicator, and a method for disinfecting a nasal passage of a patient. The applicator can generally be made by preparing a disinfecting composition as described hereinabove and then disposing the disinfecting composition on an absorbent material, such as by dipping, soaking the absorbent material with the disinfecting composition, spraying the disinfecting composition on the absorbent material, etc. The disinfecting composition itself can generally be made by combining components thereof at any suitable temperature.

After making an applicator, the prepared applicator can be placed in a container (such as container 4, illustrated in FIG. 1) using conventional packaging equipment. Any suitable number of applicators, such as two swabs, may be placed within a container. The container can then be sealed to preserve the integrity of the applicator.

In use, the disinfecting composition can generally be used to cleanse a nasal passage of a patient in any useful manner. A method of disinfecting a nasal passage of a patient can generally comprise applying a disinfecting composition as described hereinabove to the nasal passage prior to surgery. This step may be performed generally any time prior to surgery, such as within 1 hour; within 30 minutes, or within 15 minutes, prior to surgery. The method of disinfecting a nasal passage of a patient can generally comprise spraying a disinfecting composition into a nasal passage of a patient rather than using a swab.

In some embodiments, a method of disinfecting a nasal passage of a patient comprises providing a packaged applicator system (e.g. as illustrated in FIGS. 1 and 2) and after removal of an applicator from the sealed container, inserting the applicator into a nasal passage of the patient. This step may be performed by the patient or by the clinician. The method of disinfecting a nasal passage of a patient can further comprise opening a sealed container and removing an applicator from the sealed container.

The following examples are provided to illustrate the present disclosure and should not be construed as limiting in scope.

### Example 1

A disinfecting composition was made by combining 59.13 wt % of ethyl alcohol (190 proof (54.65 wt % ethanol and 4.48 wt % water)) 2.86 wt % of isopropyl alcohol, 30.54 wt % of castor oil USP, 1.00 wt % of kiwi blossom fragrance, 5.00 wt % of purified water USP, 0.80 wt % of glyceryl laurate, 0.50 wt % of benzyl alcohol, 0.10 wt % of polyaminopropyl biguanide (20%), 0.05 wt % of menthol USP, 0.01 wt % of BHT NF, and 0.01 wt % ascorbyl palmitate NF.

The obtained disinfecting composition was then applied to an in-vitro culture of MRSA. The disinfecting composition provided log₁₀ reductions of MRSA of 2.32 after fifteen seconds, 6.36 after eight hours, and 7.21 after twelve hours.

### Example 2

Two swabs are dipped in the disinfecting composition of Example 1. Each swab comprises a swab stick and an absorbent material, and after dipping the disinfectant composition is disposed on the absorbent material. The swabs are packaged in a sealed container as shown in FIG. 1 to form a packaged applicator system.

### Example 3

The swabs of the packaged applicator system of Example 2 are removed from their package and used to cleanse the nasal passages of a patient.

It is thus seen that the above disclosure provides a disinfecting composition, an applicator, and a packaged applicator system, as well as a method of disinfecting nasal passages of a patient.

All percentages stated herein are weight percentages.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or language describing an example (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. An applicator comprising:
an absorbent material, and
a disinfecting composition disposed on the absorbent material, the disinfecting composition comprising:
at least one C₁-C₃ alcohol in an amount of at least 40%, and
an emollient in amount effective to reduce a stinging sensation produced by contacting a wall of a nasal passage with the alcohol, the emollient being miscible in the alcohol.

2. An applicator according to claim 1, the emollient comprising castor oil.

3. An applicator according to claim 1, the alcohol comprising ethyl alcohol.

4. An applicator according to claim 1, the alcohol comprising ethyl alcohol and the emollient comprising castor oil.

5. An applicator according to claim 4, the alcohol further comprising a denaturant.

6. An applicator according to claim 5, the denaturant comprising isopropyl alcohol.

7. An applicator according to claim 1, the disinfecting composition further comprising a cooling agent.

8. An applicator according to claim 7, the cooling agent comprising menthol.

9. An applicator according to claim 1, the disinfecting composition further comprising an additional emollient.

10. An applicator according to claim 9, the additional emollient comprising glyceryl laurate.

11. An applicator according to claim 1, the disinfecting composition further comprising a preservative, the preservative comprising one or more of benzyl alcohol and polyaminopropyl biguanide.

12. An applicator according to claim 1, the disinfecting composition further comprising one or more of BHT and vitamin C.

13. An applicator according to claim 1, the disinfecting composition comprising ethyl alcohol, castor oil, isopropyl alcohol, and water.

14. An applicator according to claim 1, the disinfecting composition comprising ethyl alcohol, castor oil, water, isopropyl alcohol, glyceryl laurate, benzyl alcohol, polyaminopropyl biguanide, and menthol.

15. An applicator according to claim 1, the disinfecting composition comprising ethyl alcohol in an amount ranging from 50 to 70% and castor oil in an amount ranging from 20 to 40%.

16. An applicator according to claim 15, the disinfecting composition comprising water in an amount ranging from 1 to 15%, isopropyl alcohol in an amount ranging from 1 to 7%, and glyceryl laurate in an amount ranging from 0.1 to 1.5%,

17. An applicator according to claim 16, further comprising menthol in an amount ranging from 0.01 to 0.15%.

18. An applicator according to claim 1, the disinfecting composition comprising a single-phase solution.

19. An applicator according to claim 1, the applicator comprising a swab including the absorbent material and a swab stick.

20. A packaged applicator system comprising:
a sealed container enclosing two swabs, each swab comprising a swab stick and an absorbent material and
a disinfecting composition disposed on the absorbent material, the disinfecting composition comprising:
at least one C₁-C₃ alcohol in an amount of at least 40%, and
an emollient in amount effective to reduce a stinging sensation produced by contacting the wall of the nasal passage with the alcohol, the emollient being miscible in the alcohol.

21. A packaged applicator system according to claim 20, the disinfecting composition comprising ethyl alcohol, castor oil, water, isopropyl alcohol, glyceryl laurate, benzyl alcohol, polyaminopropyl biguanide, and menthol.

22. A packaged applicator system according to claim 20, the disinfecting composition comprising ethyl alcohol in an amount ranging from 50 to 70% and castor oil in an amount ranging from 20 to 40%.

23. A packaged applicator system according to claim 22, the disinfecting composition comprising water in an amount ranging from 1 to 15%, isopropyl alcohol in an amount ranging from 1 to 7%, and glyceryl laurate in an amount ranging from 0.1 to 1.5%.

24. A packaged applicator system according to claim 23, further comprising menthol in an amount ranging from 0.01 to 0.15%.

25. A packaged applicator system according to claim 20, the container comprising one or more of a polymeric film and foil.

26. A packaged applicator system according to claim 25, the container comprising separate compartments each including a single swab.

27. A method of disinfecting a nasal passage of a patient, comprising:
providing the packaged applicator system of claim 20; and
after removal of an applicator from the sealed container, inserting the applicator into the nasal passage of the patient.

28. A method according to claim 27, comprising opening the sealed container and removing an applicator from the sealed container.
